(19) United States
(12) Patent Application Publication
(10) Pub. No.: US 2005/0281760 A1
(43) Pub. Date: Dec. 22, 2005

(54) Dental materials and interdental cleaners

(76) Inventor: Deborah Overoyen, Doha (QA)

Correspondence Address: FISH & RICHARDSON P.C., PO BOX 1022, MINNEAPOLIS, MN, 55440-1022, US

(21) Appl. No.: 10/872,689

(22) Filed: Jun. 21, 2004

(51) Int. Cl. A61K 7/26

(52) U.S. Cl. 424/58

(57) ABSTRACT

The invention provides for dental materials and interdental cleaners that contain a S. persica extract.

### TECHNICAL FIELD

This invention relates to dental compositions, and more particularly to dental materials and interdental cleaners made with or containing Salvadora persica.

### BACKGROUND

Dental materials are usually combinations of silane-coated inorganic filler particles and a dimethacrylate resin. The filler particles can be barium silicate glass, quartz, or zirconium silicate, usually combined with 5-10% (by weight) of very small-sized particles (˜0.04 μm) of colloidal silica. Therefore, dental materials usually are a blend of glass or ceramic particles dispersed in a polymerizable synthetic organic resin matrix. The polymer materials are blended together with the finely divided inorganic material such as a barium aluminosilicate glass or other glass compositions having an effective amount of radiopaque oxide that renders the resultant glass radiopaque to x-rays.

Interdental cleaners such as floss, toothpicks, or plaque removers can be used to clean the surfaces of teeth that are not readily accessible with a toothbrush. The purpose of using interdental cleaners is to dislodge and remove any food material that has accumulated at the interproximal surfaces, and to dislodge and remove the growth of bacterial material (plaque) on the teeth or the superimposed calculus that has accumulated on the teeth since the previous cleaning.

### SUMMARY

The invention provides for dental materials or one or more components thereof that include a S. persica extract. The invention also provides for interdental cleaners that include a S. persica extract or that are made from S. persica.

In one aspect, the invention provides for a dental material or a component thereof that includes a S. persica extract. Dental materials can include, for example, fillers, sealants, adhesives, cements, polishing paste, restorative agents, and reconstructive agents. In some embodiments, a dental material can further include fluoride and/or anti-microbial agents.

In yet another aspect, the invention provides for an article of manufacture that includes a S. persica extract and instructions for adding the extract to a dental material or a component thereof.

In another aspect, the invention provides for an interdental cleaner that includes a S. persica extract or an interdental cleaner made from S. persica. Representative interdental cleaners include toothpicks, floss, and plaque removers. In addition to fabricating an interdental cleaner from S. persica, an interdental cleaner of the invention can be coated with an extract or can be impregnated with an extract. An interdental cleaner of the invention can further include one or more flavors, one or more vitamins, one or more minerals, and/or one or more medicines.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is a schematic of one embodiment of a plaque remover.

### DETAILED DESCRIPTION

The invention provides for dental materials or one or more components thereof that include a S. persica extract. The invention also provides for interdental cleaners that are made from S. persica and/or include a S. persica extract. The dental materials and interdental cleaners provided herein can be used in any specialized area of dentistry such as periodontal dentistry, surgical dentistry, pediatric dentistry, endodontic dentistry, prosthodontic dentistry, or dentistry specializing in temporomandibular joint (TMJ) diseases and disorders. The dental materials and interdental cleaners provided herein also can be used in patients having reduced or compromised immune systems (e.g., elderly patients, patients on renal dialysis, hospitalized patients, patients diagnosed with cancer, HIV, or AIDS, or patients with a disposition for viral, bacterial, and/or fungal infections).

S. persica and Extracts Thereof

Numerous beneficial ingredients have been identified in Salvadora persica plants. S. persica contains antibacterial acidic inhibitors that fight tooth decay and prevent diarrhea. Antibacterial acidic inhibitors are natural disinfectants and can be used to stop bleeding. They disinfect the gums and teeth and close any microscopic cuts that may exist in the gums. Another ingredient, a mustard-like substance, fights decay in the mouth and kills germs. S. persica also contains minerals such as sodium chloride, potassium, sodium bicarbonate, and calcium oxides, which clean the teeth. S. persica also contains natural scented oils that add taste and give the mouth a nice smell. In addition, S. persica contains enzymes that prevent the buildup of plaque that causes gum disease and loss of teeth. Further, S. persica contains other anti-decay and anti-germ ingredients that decrease the number of bacteria in the mouth.

Extraction is a process whereby the desired constituents (e.g., the beneficial ingredients) of a plant or plant part are removed using a solvent or other means. To produce an extract, plant material is usually first cleaned and dried if necessary. Drying can be done naturally (e.g., by air drying) or artificially (e.g., using warm-air fans or conveyor dryers). The plant material then can be ground, cut, or shredded using, for example, hammer action, pressure, friction or impact cutting. Methods of removing the desired constituents from the plant material include, but are not limited to, organic solvent extraction, supercritical gas extraction, and steam distillation.

There are a number of procedures for organic solvent extraction, including maceration (soaking and agitating the plant material with a solvent), percolation (repeated rinsing of the plant material with a solvent), and countercurrent extraction (continuous flow of a solvent in the opposite direction as the plant material). Representative solvents include, but are not limited to, ethanol, benzene, toluene and ether. Aqueous extracts, such as decoctions (boiling the plant material, generally used for hard tissues), infusions (steeping the plant material, generally used for soft tissues) or macerations, can also be produced, although microbial contamination can be a concern with aqueous extraction methods.

Methods of producing extracts containing one or more active components also are provided by the invention. For example, fractionating an extract by traditional solvent extraction employs partitioning of a solute between two immiscible phases, typically an organic phase (e.g., n-hexane, methylene dichloride, ethyl acetate or n-butanol) and an aqueous phase. Rapid extraction kinetics and the ability to utilize a number of different diluents, extractants, and aqueous phases makes solvent extraction a powerful separation method. In addition, numerous other separation procedures can be employed to further purify desired components or remove unwanted or contaminating components, including decanting, filtration, sedimentation, centrifugation, heating, adsorption, precipitation, chromatography, or ion exchange. The resulting extract containing one or more active components can be subsequently concentrated by evaporation, vaporization, lyophilization or vacuum drying. Those of skill in the art are aware of the advantages of using certain separation techniques in combination with others to increasingly partition an extract into one or more active components.

Generally, an extract of S. persica can be obtained from the bark, branches, or stem of the plant, although leaves, roots, and flowers also can be used. S. persica tissue can be chemically extracted with, for example, 80% alcohol, extracted again with, for example, ether, and run through further routine chemical procedures. Previous experiments detected trimethylamine, an alkaloid that may be salvadorine, chlorides, high amounts of fluoride and silica, sulphur, vitamin C, and small amounts of tannins, saponins, flavenoids, and sterols in a particular S. persica extract. Active components of a S. persica extract or active fractions also can include, but are not limited to, polyphenols, flavonoids, aromatic acids, metabolites, alkaloids, proteins, carbohydrates, starches, steroids, resins, elements or combinations thereof (e.g., glycoproteins) that, alone or in combination with other components, can improve oral health.

For additional information on extraction of S. persica and components of the resulting extracts, see, for example, Al lafi & Ababneh (1995, Int. Dent. J., 45:218-22); Darout et al. (2000, Ind. J. Pharm., 32:11-14); Ezmirly & El-Nasr (1981, J. Chem. Soc. Pak., 3:9-12); Gehlot and Bohra (2000, Curr. Sci. Bangalore, 78:780-1); Kamil & Ahmad (2000, Pak. J Sci. Ind. Res., 43:255-7); Almas & Al (1999, Biomed. Lett., 60:71-5); Almas & Al (1997, Biomed. Lett., 56:145-9); Al & Al (1996, Biomed. Lett., 53:225-38); Al & Idowu (1994, Microbios., 80:107-13); Galletti & Chiavari (1993, Rap. Comm. Mass. Spec., 7:651-5); Sulaiman & Ajabnoor (1986, J. Ethnopharm., 17:263-8); and U.S. Pat. Nos. 6,428,823 and 6,586,021.

Dental Materials and Interdental Cleaners

Dental materials can be used for a variety of dental treatments and restorative functions, including crown and bridge materials (either with or without an alloy substrate), filling materials, bonding materials, polishing paste, endodontic irrigating solutions, inlays, onlays, laminate veneers, dental adhesives, liner, bases, restorative treatments, pit or fissure sealants, luting agents or cements, denture base materials, core build-up materials, and orthodontic materials and sealants. In addition, dental material can include additional components such as fluoride and/or antimicrobial agents. Many dental materials are composites; i.e., have more than one component.

Filling materials are a type of dental material and are employed in dental procedures to improve mechanical properties such as compressive strength, abrasion resistance, and surface hardness. Fillers should have a suitable viscosity and low polymerization shrinkage. Fillers are usually prepared by grinding, for example, α-quartz and glass to an average particle size of a few microns to a few hundreds microns. See, for example, U.S. Pat. No. 5,356,951.

Dental cements (e.g., endodontic cements) are another type of dental material and also can be used to increase the mechanical strength of a tooth or to anchor another type of dental material such as a post. Cements can be made from, for example, glass ionomers and glass polyalkenoates. PCT Application No. WO94/23687 describes a glass ionomer which is a reaction product of a polyalkenoic acid component and a glass component.

Bonding agents are another type of dental material and are used routinely in dental procedures. For example, acid etching, or glutaraldehyde 2-hydroxyethyl methacrylate (HEMA) enhances the bonding strength of dental materials to teeth (1955, Dental Res. 34:849-854; 1984, J. Dental Res. 63:1087-1089). In addition, a number of components such as maleic acid, nitric acid, p-toluenesulfonic acid, a salt of an acid, a monomer (e.g., a polymerizable monomer) having an acidic group such as an amino acid residue (e.g., N-acryloylaniline), or a curing agent with or without an amino compound can be added to an aqueous solution of HEMA that, in turn, can be added to a dental material to improve the bonding strength. See, for example, Japanese Application Nos. JP-A-62-223289; JP-A-1-113057; JP-A-3-240712; and JP-A-4-8368; and Materials and Instruments for Dental Use, 9:65-73, 1990.

Sealants are another type of dental material. Sealants usually are made with plastic resins and are applied to the chewing surfaces of the premolars and molars. Sealants fill the depressions and grooves (pits and fissures) of the chewing surfaces of teeth. The sealant acts as a barrier, protecting enamel from plaque, acids, and bacteria.

Polishing paste (e.g., prophy paste) is a dental material used by dentists and hygienists to clean teeth (e.g., remove stains) and to polish porcelain. Polishing pastes can include abrasives (e.g., pumice, or perlite), fluoride, and one or more flavoring components. “Grit” refers generally to the size of abrasive particles. For example, a fine grit polishing paste can be used for light stain removal and amalgam polishing; a medium grit polishing paste can be used for normal cleaning and polishing; a coarse grit polishing paste can be used for medium to heavy stain and plaque removal; and a heavy grit polishing paste can be used for removal of heavy stain (e.g., tobacco) and plaque deposits.

Alloys (i.e., comprising 2 or more different metals) are another dental material used in dentistry because alloying strengthens a metal. Several properties have a bearing on the clinical performance of prosthodontic alloys including, but not limited to yield strength (e.g., the amount of stress required to produce a change in length of an alloy), hardness (e.g., enough to resist wear but not too hard to damage the opposing dentition), modulus of elasticity (e.g., the amount of rigidity or stiffness of an alloy), proportional limit (e.g., the amount of stress required to produce permanent deformation of an alloy), and corrosion (e.g., the amount of surface deterioration). Dental alloys are currently classified based on their physical properties (e.g., hardness or yield strength) or their composition.

Endodontic irrigating solutions are another dental material used routinely in dentistry. Endodontic irrigating solutions aid cleaning, increase the permeability of dentin, enlarge dentinal tubules, and allow instrumentation of atresic canals for cleaning. Both the chemical and mechanical actions of irrigating solutions are effective in removing debris and in lubricating endodontic instruments. Representative endodontic irrigating solutions include, but are not limited to, sodium hypochlorite (e.g., 0.5%), chlorhexidine (e.g., 0.2%-2%), saline (e.g., 0.9%), NaOCl (e.g., 2.5%), H₂O₂ (e.g., 3%), or EDTA (e.g., 17%).

Dental appliances are another type of dental material. Dental appliances include devices for correcting, supplementing, or replacing teeth (e.g., orthodontic retainers, biteplates, and dentures) as well as appliances used for other purposes (e.g., to treat obstructive sleep-disordered breathing (OSDB such as snoring, apnea, and upper airway resistance syndrome by preventing vibration and collapse of the upper airway, or to reduce the amount of food an individual eats).

Dentures including denture bases, denture cleaners, and denture liners are another type of dental material. A denture base is the portion of the denture that overlies the soft tissue and supports the supplied teeth, and is supported in turn by abutment teeth or the residual alveolar ridge. Dental bases can be made with one or more resins, metals, or combinations thereof, and also can contain pigments for natural coloring. Dental cleaners are used to remove stains, odor, and bacteria from dentures. Dentures can be soaked in or brushed with a cleaner. Denture liners are placed between the denture and soft tissue to reduce or eliminate pain due to tissue compression. Denture liners can be made from rubber or silicone compounds.

Breath mints, sprays, drops, films, or papers also are dental materials. Such materials generally reduce odor-causing bacteria in the mouth and, therefore, typically do not contain sugar. Chewing gum also is a dental material. Chewing gum can clean teeth after a meal, freshen breath, moisten mouth, and/or deliver vitamins or medicines. Chewing gums can include a base (e.g., latex), sweeteners, softeners, flavorings or colorings, and preservatives.

Some dental materials require two or more components (e.g., composite dental materials). Individual components of a composite material can be combined by a manufacturer. Additionally or alternatively, components of a composite material can be mixed by practitioners, for example, to cause polymerization of the material. Resins, monomers (e.g., a polymerizable monomer), a polymerization initiator, metal alloys, and/or ceramics, for example, can be used in composite dental materials. Composite dental materials can include organic material, inorganic material, or both. A composite material also can include a silane-coupling agent, which can improve the polymerization of a composite material.

Interdental cleaners can include a variety of objects, such as dental floss, toothpicks, and plaque-removers. Interdental cleaners are for probing and/or scraping the teeth, usually between teeth, to remove plaque that remains after brushing.

Dental floss is usually made from a plurality (e.g., 50-500) of monofilaments (e.g., nylon monofilaments) of approximately 0.002-0.004 inches in diameter joined together by, for example, weaving, pleating, or twisting to make a strand of dental floss of a suitable size to be inserted between the teeth. The dental floss may be round or flat in cross-section. Most floss sold at retail today is also “waxed” to assist penetration to interproximal regions of teeth. Floss also can include particles made of abrasive or polishing materials (e.g., aluminum oxide) integrated into the monofilaments (e.g., during manufacture and/or prior to extrusion).

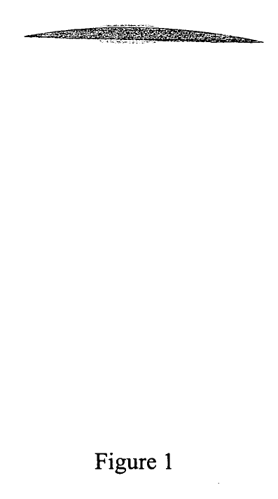

Many people in the world do not floss their teeth. Instead, they use plaque removers or toothpicks to clean their teeth. Plaque removers and toothpicks are similar, but toothpicks are usually round or flat and may be tapered, while plaque removers are thin, pliable, wedge-shaped sticks (see FIG. 1) that are often made of basswood. Plaque removers are able to get between the teeth to remove tough, out-of-the-way plaque. In addition, the wedge shape is gentle on gums. If used properly, plaque removers or toothpicks can be an effective alternative to flossing.

Dental Materials and Interdental Cleaners Made with or Containing S. persica

With respect to dental materials, a S. persica extract can be added during the manufacturing process of one or more components of a dental material, or the extract can be added to a dental material by a practitioner (e.g., a dentist, or a hygienist) prior to applying the material to a tooth.

With respect to interdental cleaners, an interdental cleaner can be fabricated out of S. persica sticks using traditional methods (e.g., see, for example, U.S. Pat. No. 1,613,623; or current methods of veneering or extruding), or an interdental cleaner fabricated out of traditional materials (e.g., birch, willow, maple, or plastic) can be coated with a S. persica extract (e.g., sprayed on) or impregnated with the extract (e.g., by soaking). Interdental cleaners also can be flavored, and/or can contain vitamins, minerals and/or medicines (e.g., antibiotics).

Addition of a S. persica extract preferably does not significantly change the properties of a dental material or an interdental cleaner. For example, addition of a S. persica extract to a dental material or an interdental cleaner optimally should not significantly change, for example, the mechanical properties (e.g., the ability to withstand gliding occlusal pressure upon mastication), the durability, the thermal expansion coefficient, the biocompatibility (e.g., non-toxicity), the insolubility, the low water absorbing property, and the amount of shrinkage upon polymerization and/or curing. Addition of a S. persica extract also preferably does not significantly alter the releasability of components such as antimicrobial agents or fluoride from a dental material or an interdental cleaner, and optimally does not significantly alter the ability of the material or cleaner to prevent the growth of cariogenic bacteria. Addition of a S. persica extract preferably does not significantly alter the ability to handle and apply a dental material as a paste prior to polymerization, and further, preferably does not significantly alter the optical properties such as color tone, color adaptation, coloring resistance, the transparency, and the surface smoothness and glossiness achieved after polishing.

As discussed above, a S. persica extract can be introduced into a dental material or a component thereof by the manufacturer, and provided to practitioners in an article of manufacture (e.g., a kit) with instructions for use. Alternatively, a S. persica extract can be provided in a container to a practitioner with appropriate instructions for mixing the extract with the appropriate dental material or components(s) thereof. Instructions to a practitioner for using a S. persica can include appropriate amounts for adding to a dental material or a component thereof and/or amounts for applying to one or more teeth.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A dental material or a component thereof, wherein said dental material or a component thereof comprises a S. persica extract.

2. The dental material of claim 1, wherein said dental material is selected from the group consisting of a filler, a sealant, an adhesive, an alloy, a polishing paste, a bonding agent, an endodontic irrigating solution, a cement, a dental appliance, a restorative agent and a reconstructive agent.

3. The dental material of claim 2, wherein said appliance is dentures.

4. The dental material of claim 1, wherein said dental material is selected from the group consisting of a breath mint, a breath spray, breath drops, breath films, and breath papers.

5. The dental material of claim 1, wherein said dental material or said component thereof further comprises fluoride and/or an anti-microbial agent.

6. An interdental cleaner, wherein said cleaner comprises a S. persica extract.

7. The interdental cleaner of claim 6, wherein said interdental cleaner is selected from the group consisting of a toothpick, floss, and a plaque remover.

8. The interdental cleaner of claim 6, wherein said interdental cleaner is coated with said extract.

9. The interdental cleaner of claim 6, wherein said interdental cleaner is impregnated with said extract.

10. The interdental cleaner of claim 6, wherein said interdental cleaner further comprises one or more flavors, one or more vitamins, one or more minerals, and/or one or more medicines.

11. An interdental cleaner made from S. persica.

12. An article of manufacture comprising a S. persica extract and instructions for adding the extract to a dental material or a component thereof.
